(19) United States
(12) Patent Application Publication
(10) Pub. No.: US 2026/0263160 A1
(43) Pub. Date: Sep. 10, 2026

(54) ABLATION AUTOMATED NAVIGATION METHOD AND SYSTEM

(71) Applicant: Hunan Cancer Hospital, Changsha (CN)

(72) Inventors: Wen LU, Changsha (CN); Hua XIANG, Changsha (CN); Qinqin CHENG, Changsha (CN); Zhengdi SHE, Changsha (CN); Guowen LI, Changsha (CN); Yang ZHAO, Changsha (CN)

(21) Appl. No.: 19/332,574

(22) Filed: Sep. 18, 2025

(30) Foreign Application Priority Data
Mar. 6, 2025 (CN) 202510262534.X

(51) Int. Cl. A61B 34/20; A61B 18/00; A61B 34/10; G06N 7/08

(52) U.S. Cl. CPC A61B 34/20; A61B 18/00; A61B 34/10; G06N 7/08; A61B 2018/00577; A61B 2034/105; A61B 2034/2055

(57) ABSTRACT

An ablation automated navigation method includes: setting hard constraint conditions and soft constraint conditions for an ablation path; initializing multiple preliminary ablation paths that meet the hard constraint conditions; calculating a risk index of each of the preliminary ablation paths; sorting each of the preliminary ablation paths in ascending order of the risk index, and selecting a first preset number of preliminary ablation paths with top rankings as initial solutions of a seagull optimization algorithm; optimizing the ablation path through the seagull optimization algorithm to determine multiple candidate ablation paths; sorting each of the candidate ablation paths in ascending order of fitness, and selecting a second preset number of candidate ablation paths with top rankings for organ damage assessment; determining an optimal ablation path from the candidate ablation paths; performing ablation navigation according to a needle insertion position, angle and depth determined by the optimal ablation path.

### CROSS-REFERENCE TO RELATED APPLICATIONS

This application claims priority to a Chinese Application No. 202510262534.X, filed on Mar. 6, 2025, titled “Ablation Automated Navigation Method and System”, the entire contents of which are hereby incorporated herein by reference in their entirety.

### TECHNICAL FIELD

The present application relates to the technical field of computer-aided medicine, and in particular to an ablation automated navigation method and system.

### BACKGROUND

Ablation automated navigation improves the precision and safety of ablation therapy through accurate path planning and real-time feedback, reduces doctors' operational errors, avoids damage to normal tissues, and enhances treatment effects. It not only shortens the operation time and promotes the rapid recovery of patients, but also reduces the operational pressure on doctors and improves the operation efficiency through personalized treatment plans and decision support. It is an important technology for improving treatment quality and patient experience in modern medical care.

The current ablation navigation technology mainly relies on medical images to complete the ablation path planning. However, when dealing with cases of multiple tumors, deeply located tumors or tumors with complex locations, the current ablation navigation technology has limited ability in ablation path planning. It is difficult to conduct a comprehensive risk assessment of the ablation path and cannot evaluate the potential damage of the path to surrounding tissues and organs, which may lead to improper selection of puncture paths and cause irreversible damage or complications.

### SUMMARY

To solve the technical problems existing in the current ablation navigation technology, such as limited ability in ablation path planning when dealing with cases involving multiple tumors, deeply located tumors or tumors with complex locations, difficulty in conducting a comprehensive risk assessment of the ablation path, inability to evaluate the potential damage of the path to surrounding tissues and organs, which may lead to improper selection of puncture paths and cause irreversible damage or complications, the present application provides an ablation automated navigation method and system.

The technical solutions provided by the embodiments of the present application are as follows:

### First Aspect:

S1: setting hard constraint conditions and soft constraint conditions for an ablation path;
S2: based on the hard constraint conditions, initializing multiple preliminary ablation paths that meet the hard constraint conditions;
S3: based on the soft constraint conditions, calculating a risk index of each of the preliminary ablation paths;
S4: sorting each of the preliminary ablation paths in ascending order of the risk index, and selecting a first preset number of preliminary ablation paths with top rankings as initial solutions of a seagull optimization algorithm;
S5: optimizing the ablation path through the seagull optimization algorithm to determine multiple candidate ablation paths;
S6: sorting each of the candidate ablation paths in ascending order of fitness, and selecting a second preset number of candidate ablation paths with top rankings for organ damage assessment;
S7: determining an optimal ablation path from the candidate ablation paths according to an organ damage assessment result;
S8: performing ablation navigation according to a needle insertion position, angle and depth determined by the optimal ablation path.

### Second Aspect:

An ablation automated navigation system provided by an embodiment of the present application includes:
a processor;
a memory, where computer-readable instructions are stored on the memory, and when the computer-readable instructions are executed by the processor, the ablation automated navigation method as described in the first aspect is implemented.

### Third Aspect:

A computer-readable storage medium provided by an embodiment of the present application has a computer program stored thereon, and when the program is executed by a processor, the ablation automated navigation method as described in the first aspect is implemented.

The beneficial effects brought by the technical solutions provided by the embodiments of the present application include at least:

In the embodiment of the present application, hard constraint conditions, soft constraint conditions, the seagull optimization algorithm and organ damage assessment are adopted, and the optimal ablation path is determined through multi-stage path optimization. It can deal with cases involving multiple tumors, deeply located tumors or tumors with complex locations, conduct a comprehensive risk assessment of the ablation path, and evaluate the potential damage of the path to surrounding tissues and organs. This can ensure that the finally selected path not only has a low risk index, but also avoids damage to normal organs to the greatest extent, ensures the safety and effectiveness of the ablation process, and improves the accuracy of path selection and the efficiency of ablation navigation.

### BRIEF DESCRIPTION OF THE DRAWINGS

To more clearly illustrate the technical solutions in the embodiments of the present application, the accompanying drawings required for describing the embodiments will be briefly introduced below. Obviously, the accompanying drawings in the following description are only some embodiments of the present application. For those skilled in the art, other accompanying drawings can also be obtained based on these accompanying drawings without exerting creative efforts.

FIG. 1 is a schematic flowchart of an ablation automated navigation method provided by an embodiment of the present application.

FIG. 2 is a schematic structural diagram of an ablation automated navigation system provided by an embodiment of the present application.

### DETAILED DESCRIPTION OF THE EMBODIMENTS

Below is a description of the technical solutions in the present application with reference to the accompanying drawings.

In the embodiments of the present application, words such as “exemplarily” and “for example” are used to provide examples, illustrations, or explanations. Any embodiment or design solution described as an “example” in the present application shall not be construed as being more preferred or advantageous than other embodiments or design solutions. Rather, the use of the word “example” is intended to present concepts in a concrete manner. Furthermore, in the embodiments of the present application, the meaning expressed by “and/or” may include both of the two options, or either one of the two options.

In the embodiments of the present application, “image” and “picture” may sometimes be used interchangeably; it should be noted that when their differences are not emphasized, the meanings they intend to convey are consistent. “Of”, “relevant” and “corresponding” may sometimes be used interchangeably; it should be noted that when their differences are not emphasized, the meanings they intend to convey are consistent.

To make the technical problems to be solved, technical solutions, and advantages of the present application clearer, a detailed description will be given below with reference to the accompanying drawings and specific embodiments.

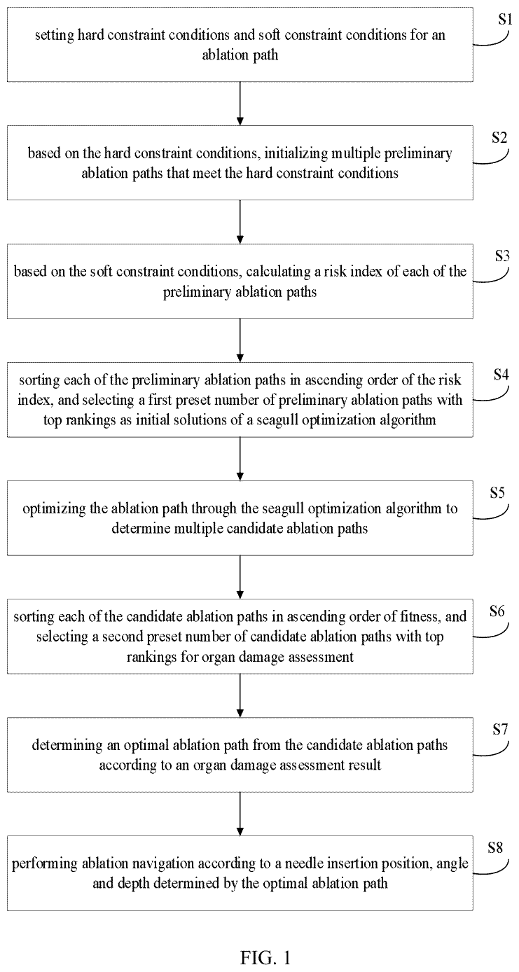

With reference to FIG. 1 in the specification, a schematic flowchart of an ablation automated navigation method provided by an embodiment of the present application is shown.

An embodiment of the present application provides an ablation automated navigation method, which can be implemented by an ablation automated navigation device. The ablation automated navigation device may be a terminal or a server. The processing flow of the ablation automated navigation method may include the following steps:
S1: Setting hard constraint conditions and soft constraint conditions for the ablation path.

The hard constraint conditions refer to the basic requirements that must be met in ablation path planning, mainly including restrictions such as avoiding risky structures (such as important organs, blood vessels, etc.), puncture depth, puncture angle, and needle length. These hard constraint conditions ensure the basic safety and feasibility of the ablation process and serve as an essential prerequisite for path planning.

Optionally, the hard constraint conditions specifically include: obstacle avoidance constraint, puncture depth constraint, puncture angle constraint, and needle length constraint.

Obstacle avoidance constraint: the ablation path must avoid risky structures.

The risky structures may be important organs, blood vessels, etc.

It should be noted that the obstacle avoidance constraint requires the ablation path to avoid all risky structures, which minimizes damage to surrounding healthy tissues and important organs during the ablation process, reduces the incidence of surgical complications, and ensures the safety of the ablation process. By preventing the puncture path from colliding with risky structures, the risk of severe complications such as bleeding and infection can be reduced.

Puncture depth constraint: the puncture depth of the ablation path must be greater than a set depth threshold.

Those skilled in the art can set the value of the depth threshold according to actual conditions, and the present application does not impose limitations thereon.

It should be noted that the puncture depth constraint ensures that the puncture depth of the ablation needle is greater than the set depth threshold, which can ensure that the ablation needle can reach the depth where the tumor is located and perform effective treatment. If the depth is insufficient, the ablation needle may fail to accurately reach the tumor, affecting the treatment effect. Setting an appropriate depth threshold helps avoid excessive puncture or damage to surface tissues and ensures the effectiveness and safety of the ablation area.

Puncture angle constraint: the puncture angle at which the ablation path intersects the surface of the ablation organ must be greater than a set angle threshold.

Those skilled in the art can set the value of the angle threshold according to actual conditions, and the present application does not impose limitations thereon.

It should be noted that the puncture angle constraint requires the puncture angle at which the ablation path intersects the surface of the ablation organ to be greater than the set angle threshold, which can ensure that the ablation needle enters the ablation organ at an appropriate angle, thereby better avoiding blood vessels and other key structures of the ablation organ and reducing the risk of bleeding and other complications. In addition, an appropriate angle helps ensure the accurate positioning of the ablation needle and improves the treatment effect.

Needle length constraint: the length of the ablation path is less than or equal to the maximum length of the needle.

It should be noted that the needle length constraint ensures that the length of the ablation path is less than or equal to the maximum length of the needle, which guarantees the operability of the ablation path and avoids situations where the needle is not suitable for actual treatment due to an excessively long path. By limiting the path length, it can be ensured that the ablation needle can be smoothly inserted into the target position, preventing the accuracy and safety of the treatment from being affected by insufficient or excessive length.

The soft constraint conditions are optimization requirements for ablation path planning, aiming to further improve the safety and efficiency of the path on the basis of meeting the hard constraint conditions. The soft constraints include requirements such as avoiding the path from passing through risky structures as much as possible, increasing the puncture angle, reducing the puncture depth, and reducing the number of CT layers passed through. These soft constraint conditions do not have to be strictly met, but they can optimize path selection, reduce damage to normal tissues, and improve the overall effect of the ablation process and the patient's recovery speed.

Optionally, the soft constraint conditions specifically include: a first soft constraint, a second soft constraint, a third soft constraint, and a fourth soft constraint.

First soft constraint: the ablation path should be as far away from risky structures as possible.

It should be noted that by avoiding risky structures near the ablation organ to the greatest extent, the risk of damage to surrounding normal tissues and organs caused by the puncture path can be effectively reduced. Reducing contact with these important structures can significantly reduce the incidence of postoperative complications, such as bleeding, infection, and organ function damage, thereby improving the safety of the treatment.

Second soft constraint: the puncture angle at which the ablation path intersects the surface of the ablation organ should be as large as possible.

It should be noted that choosing a larger puncture angle can ensure the stability of the puncture, and can ensure that the ablation needle better avoids key structures such as blood vessels and bile ducts of the ablation organ, reducing damage to these sensitive areas during the puncture process. A larger puncture angle can usually help ensure that the insertion path of the ablation needle is more perpendicular to the surface of the ablation organ, avoiding excessive puncture of the outer tissue of the ablation organ, thereby ensuring the accuracy and effectiveness of the treatment.

Third soft constraint: the depth of the ablation path should be as shallow as possible.

It should be noted that choosing a shallower ablation path can reduce the puncture depth into the liver surface and other tissues, lowering the risk of damage to the liver surface and other adjacent organs during the operation. A shallower path also helps reduce the difficulty of operating the ablation needle, reduces trauma to the patient, and at the same time reduces the occurrence of postoperative complications such as bleeding and infection.

Fourth soft constraint: the number of CT layers passed through by the ablation path should be as small as possible.

It should be noted that reducing the number of CT layers passed through can shorten the patient's radiation exposure time during the operation and reduce the potential health risks caused by radiation. In addition, fewer CT layers mean that the path planning is more concise, which reduces path complexity, thereby lowering the difficulty of the operation and improving the efficiency and accuracy of the treatment.

S2: Based on the hard constraint conditions, initializing multiple preliminary ablation paths that meet the hard constraint conditions.

Specifically, a path search algorithm (such as A* algorithm, Dijkstra algorithm, etc.) can be used to perform path search in the three-dimensional model to ensure that multiple preliminary ablation paths meeting the hard constraint conditions are found.

S3: Based on the soft constraint conditions, calculating the risk index of each preliminary ablation path.

In a possible implementation manner, S3 specifically includes sub-steps S301 to S303:

S301: Counting the distance between each preliminary ablation path and the risky structures, the puncture angle, the puncture depth, and the number of CT layers passed through.

S302: Performing normalization processing on the distance between each preliminary ablation path and the risky structures, the puncture angle, the puncture depth, and the number of CT layers passed through, and determining the risky structure risk value, puncture angle risk value, puncture depth risk value, and the risk value of the number of CT layers passed through by the preliminary ablation path.

It should be noted that in path planning, various soft constraint conditions usually have different dimensions and value ranges. For example, the distance of the path may be in centimeters, the angle in degrees, and the depth in millimeters or centimeters. The differences between these values will affect their comparison and integration in calculations. Normalization can convert them into the same value range, allowing them to be weighted and compared on the same basis.

Optionally, S302 specifically includes:

S3021: Performing normalization processing on the distance between each preliminary ablation path and the risky structures, and determining the risky structure risk value of the preliminary ablation path:

$${f}_{d i} = \frac{{d}_{max} - {d}_{i}}{{d}_{max} - {d}_{min}} ;$$

f_{di} represents the risky structure risk value of the i-th preliminary ablation path, dᵢ represents the distance between the i-th preliminary ablation path and the risky structures, dₘₐₓ represents the maximum distance from the risky structures among all preliminary ablation paths, and dₘᵢₙ represents the minimum distance from the risky structures among all preliminary ablation paths.

S3022: Performing normalization processing on the puncture angle of each preliminary ablation path, and determining the puncture angle risk value of the preliminary ablation path:

$${f}_{a i} = \frac{{a}_{max} - {a}_{i}}{{a}_{max} - {a}_{min}} ;$$

fₐᵢ represents the puncture angle risk value of the i-th preliminary ablation path, aᵢ represents the puncture angle of the i-th preliminary ablation path, dₘₐₓ represents the maximum puncture angle among all preliminary ablation paths, and dₘᵢₙ represents the minimum puncture angle among all preliminary ablation paths.

S3023: Performing normalization processing on the puncture depth of each preliminary ablation path, and determining the puncture depth risk value of the preliminary ablation path:

$${f}_{h i} = \frac{{h}_{i} - {h}_{min}}{{h}_{max} - {h}_{min}} ;$$

fₕᵢ represents the puncture depth risk value of the i-th preliminary ablation path, hᵢ represents the puncture depth of the i-th preliminary ablation path, hₘₐₓ represents the maximum puncture depth among all preliminary ablation paths, and hₘᵢₙ represents the minimum puncture depth among all preliminary ablation paths.

S3024: Performing normalization processing on the number of CT layers passed through by each preliminary ablation path, and determining the risk value of the number of CT layers passed through by the preliminary ablation path:

$${f}_{θ i} = \frac{{θ}_{i} - {θ}_{min}}{{θ}_{max} - {θ}_{min}} ;$$

f_{θi} represents the risk value of the number of CT layers passed through by the i-th preliminary ablation path, θᵢ represents the number of CT layers passed through by the i-th preliminary ablation path, θₘₐₓ represents the maximum number of CT layers passed through among all preliminary ablation paths, and θₘᵢₙ represents the minimum number of CT layers passed through among all preliminary ablation paths.

S303: Calculating the risk index of the preliminary ablation path based on the risky structure risk value, puncture angle risk value, puncture depth risk value, and the risk value of the number of CT layers passed through by the preliminary ablation path.

Optionally, the calculation method of the risk index in S303 is specifically:

$${ρ}_{i} = {μ}_{d} {f}_{d i} + {μ}_{a} {f}_{a i} + {μ}_{h} {f}_{h i} + {μ}_{θ} {f}_{θ i} ;$$

ρᵢ represents the risk index of the i-th preliminary ablation path, μ_{d} represents the fusion coefficient of the risky structure term, μₐ represents the fusion coefficient of the puncture angle term, μₕ represents the fusion coefficient of the puncture depth term, and μ_{θ} represents the fusion coefficient of the CT layer number term.

Those skilled in the art can set the values of the fusion coefficient μ_{d} of the risky structure term, the fusion coefficient pa of the puncture angle term, the fusion coefficient μₕ of the puncture depth term, and the fusion coefficient po of the CT layer number term according to actual conditions, and the present application does not impose limitations thereon.

In the present application, the calculation method of the risk index combines multiple soft constraint conditions, enabling a comprehensive assessment of the safety of each preliminary ablation path. The risk value of each factor represents the performance of the path in a specific aspect, and the comprehensive calculation of these risk values helps to obtain a more comprehensive evaluation, ensuring that path selection takes into account all key safety factors.

S4: Sorting each preliminary ablation path in ascending order of risk index, and selecting the first preset number of preliminary ablation paths with top rankings as the initial solutions of the seagull optimization algorithm.

Optionally, the first preset number is specifically 10.

S5: Optimizing the ablation path through the seagull optimization algorithm to determine multiple candidate ablation paths.

The seagull optimization algorithm is a nature-inspired algorithm that simulates the foraging behavior of seagull colonies. It optimizes the problem-solving process by simulating the cooperation and competition among seagulls in the process of searching for food. In this algorithm, each seagull represents a potential solution, and seagulls update the quality of solutions through following and attacking behaviors to find the optimal solution. The seagull optimization algorithm has global search capability and strong explorability, and can effectively solve complex optimization problems such as ablation path planning and machine learning parameter tuning. Its advantages lie in being able to find the global optimal or near-optimal solution in the solution space, with good convergence and robustness.

Optionally, the fitness function of the seagull optimization algorithm is specifically:

$$F ( l ) = {λ}_{d} {d}_{l} + {λ}_{a} {a}_{l} - {λ}_{h} {h}_{l} - {λ}_{h} {θ}_{l} ;$$

Wherein, F( ) represents the fitness function, l represents the ablation path, dₗ represents the distance between the ablation path 1 and the risky structure, λ_{d} represents the weight coefficient of the risky structure term, al represents the puncture angle of the ablation path 1, λₐ represents the weight coefficient of the puncture angle term, hₗ represents the puncture depth of the ablation path 1, λₕ represents the weight coefficient of the puncture depth term, θₗ represents the number of CT layers passed through by the ablation path 1, and λ_{θ} represents the weight coefficient of the term of the number of CT layers passed through.

Those skilled in the art can set the values of the weight coefficient λ_{d} of the risky structure term, the weight coefficient λₐ of the puncture angle term, the weight coefficient λₕ of the puncture depth term, and the weight coefficient λ_{θ} of the term of the number of CT layers passed through according to actual conditions, and the present application does not impose limitations thereon.

Specifically, the ablation path 1 can be represented in an encoded form to facilitate the search of the seagull optimization algorithm.

In the present application, through the design of such a fitness function, multiple factors of the ablation path can be comprehensively considered, enabling a comprehensive assessment of the safety and effectiveness of the path, while dynamically adjusting the optimization focus according to different clinical needs and treatment objectives.

The present application introduces a brand-new seagull optimization algorithm, and the specific method for determining multiple candidate ablation paths through the seagull optimization algorithm is as follows:
sorting each preliminary ablation path in ascending order of risk index, selecting the first preset number of preliminary ablation paths with top rankings as the initial solutions of the seagull optimization algorithm, and initializing seagull individuals. Each seagull individual represents a set of feasible model parameters, and each seagull individual is composed of multiple dimensional components, with each component representing a model parameter.

In the global search phase, avoid collision and move towards the optimal individual:

$${D}_{i}^{t} = \vert {C}_{i}^{t} + {M}_{i}^{t} \vert$$

$${C}_{i}^{t} = A \times {P}_{i}^{t}$$

$${M}_{i}^{t} = B \times ( {P}_{b e s t}^{t} - {P}_{i}^{t} ) ;$$

Wherein,

$${D}_{i}^{t}$$

represents the position of the i-th seagull individual after the global search phase in the t-th iteration,

$${C}_{i}^{t}$$

represents the position of the i-th seagull individual after anti-collision processing in the t-th iteration, A represents the control factor,

$${P}_{i}^{t}$$

represents the position of the i-th seagull individual in the t-th iteration,

$${M}_{i}^{t}$$

represents the displacement of the i-th seagull individual moving towards the optimal individual in the t-th iteration, B represents the search balance factor, and

$${P}_{b e s t}^{t}$$

represents the position of the optimal individual in the t-th iteration.

In the present application, the anti-collision processing ensures that individuals in the search process do not generate unreasonable paths or overlaps, thereby avoiding unnecessary calculations and optimization deviations.

Furthermore, through the control factor and the search balance factor, the algorithm can balance the relationship between exploration and exploitation, enabling individuals to maintain a certain degree of randomness during the search process while gradually converging towards the optimal solution. This mechanism enhances the global search capability of the algorithm, avoids falling into local optimal solutions, and at the same time improves the convergence speed and the accuracy of path optimization.

$$A = [ 1 - lg ( \frac{9 t}{T} ) ] \times {f}_{c} ;$$

Wherein, t represents the current number of iterations, T represents the maximum number of iterations, and f_{c} represents the linear descent frequency.

In the present application, the value of the control factor is gradually reduced as the number of iterations increases, so that the search process has strong explorability in the early stage and can search the solution space extensively. In the later stage, the local search capability is enhanced, and the search is gradually concentrated near the optimal solution. This dynamic adjustment can avoid premature convergence in the early stage, ensure the diversity of the search, and accelerate convergence in the later stage, improving the efficiency and stability of the algorithm.

$$B = 2 {A}^{2} {r}_{1} ;$$

Wherein, r₁ represents a random number between 0 and 1.

In the present application, the random number r₁ makes the search steps in each iteration have a certain degree of uncertainty, thereby preventing the algorithm from falling into local optimal solutions. As the control factor gradually decreases, the search balance factor is also adjusted accordingly, enhancing the flexibility of the search. In this way, the algorithm can explore more solution spaces in the early stage, and converge more concentratedly towards the optimal solution in the later stage, balancing the capabilities of global search and local optimization, and improving the efficiency and robustness of the algorithm.

In the local search phase, generate a random number r₂, and according to the random number r₂, select in parallel between the spiral search strategy and the encirclement strategy, and perform displacement in a spiral motion manner:

$$\{ \begin{matrix}{E}_{i}^{t} = {D}_{i}^{t} xyz + {P}_{best}^{t} & {r}_{2} \leq 0.5 \\ {E}_{i}^{t} = ( {D}_{i}^{t} + {P}_{best}^{t} ) xyz & {r}_{2} \leq 0.5\end{matrix}$$

$$x = r sin η$$

$$x = r cos η$$

$$z = r η$$

$$r = {ue}^{η v} ;$$

Wherein,

$${E}_{i}^{t}$$

represents the position of the i-th seagull individual after spiral motion in the t-th iteration, x represents the spiral flight coefficient in the x direction, y represents the spiral flight coefficient in the y direction, z represents the spiral flight coefficient in the z direction, r represents the radius of the spiral flight trajectory, η represents a random number between 0 and 2π, u and v represent spiral constants, and e represents the natural constant.

In the present application, by generating a random number and selecting in parallel between the spiral search strategy and the encirclement strategy, the flexibility and diversity of the search can be increased. The introduction of the spiral motion mode helps the seagull individuals expand and contract in a spiral shape in the solution space, thereby avoiding falling into local optimal solutions. This strategy can perform detailed local searches when approaching the optimal solution, and provide more exploration opportunities when far from the optimal solution, thus balancing the needs of global search and local optimization. By introducing randomness and spiral paths, the algorithm can more effectively find the optimal solution in the solution space, improving the efficiency and accuracy of the optimization process.

Performing mutation operation on each seagull individual:

$${V}_{i}^{t} = {E}_{i}^{t} + γ ( {P}_{best}^{t} - {P}_{r} ) ;$$

Wherein,

$${V}_{i}^{t}$$

represents the position of the i-th seagull individual after mutation in the t-th iteration, Pr represents a random individual, and γ represents the adaptive scale factor.

In the present application, the mutation operation can introduce new solutions based on the current solutions, making the search process more flexible, which not only enhances the exploration capability of the algorithm but also improves the convergence speed, helping to find better solutions.

$$γ = {γ}_{max} - ( {γ}_{max} - {γ}_{min} ) sin [ \frac{π}{2} {( \frac{t}{T} )}^{2} ] ;$$

Wherein, γₘₐₓ represents the maximum scale factor, γₘᵢₙ represents the minimum scale factor, and sin represents the sine function.

In the present application, the dynamic adjustment method can provide a larger mutation range in the early stage of the search, thereby enhancing the global exploration capability and preventing the algorithm from falling into local optimal solutions. In the later stage of iteration, as the scale factor gradually decreases, the algorithm gradually converges near the optimal solution and performs more refined local searches. This gradually reduced mutation range ensures a good balance between global search and local optimization, improving the search efficiency and the quality of the final solution.

Judging whether the fitness value of the mutated position is greater than that of the position before mutation. If yes, replacing the position before mutation with the mutated position; otherwise, keep the position before mutation unchanged. Updating the fitness value of each seagull individual and the global optimal individual.

Judging whether the current number of iterations reaches the maximum number of iterations.

If yes, outputting the model parameter set represented by the seagull individual with the highest current fitness; otherwise, returning to continue iteration.

In the present application, by optimizing the ablation path through the seagull optimization algorithm, the path space can be effectively explored and optimized, and multiple candidate ablation paths can be determined.

S6: Sorting each candidate ablation path in ascending order of fitness, and selecting the second preset number of candidate ablation paths with top rankings for organ damage assessment.

Optionally, the second preset number is specifically 5.

S7: Determining the optimal ablation path from the candidate ablation paths according to the organ damage assessment result.

In a possible implementation manner, S7 specifically includes sub-steps S701 to S703:

S701: Performing functional zoning on the ablation organ.

Specifically, the functional zoning of the ablation organ can be carried out with reference to current academic research results. For example, for the liver, the current academic community can divide the liver into 8 functionally independent units (i.e., Couinaud's eight segments) by using the vascular supply in the liver, which provides an anatomical basis for liver tumor surgery planning.

S702: Determining the number of functional zones passed through by each candidate ablation path, and conduct organ damage assessment based on the number of functional zones passed through. The fewer the number of functional zones passed through, the less damage to the organ; the more the number of functional zones passed through, the greater the damage to the organ.

It should be noted that by performing functional zoning on the ablation organ and conducting organ damage assessment based on the number of functional zones passed through by each candidate ablation path, it can be ensured that the ablation path minimizes the impact on the function of organs such as the liver. Fewer functional zones passed through means less damage to normal tissues, thereby reducing the risk of postoperative complications and protecting more healthy liver functional areas.

S703: Determining the candidate ablation path with the smallest number of passed functional zones as the optimal ablation path. In the present application, by selecting the path that passes through fewer functional zones as the optimal path, not only the treatment effect is optimized, but also the safety is improved, and the damage to important functional areas during the ablation process is reduced. Ultimately, it can ensure that the ablation treatment is more accurate, effective and safe, and improve the patient's treatment effect and recovery speed.

S8: Performing ablation navigation according to the needle insertion position, angle and depth determined by the optimal ablation path.

In a possible implementation manner, S8 specifically includes:

S801: Pasting multiple markers on the surface of the patient's abdominal skin.

S802: Based on the multiple markers, performing preliminary registration between the three-dimensional model constructed based on preoperative images and the patient's point cloud:

$${L}_{intra} = {T}_{initial} \cdot {T}_{Marker}^{Tra} \cdot {T}_{US}^{Marker} \cdot {L}_{pre} ;$$

Wherein, Lᵢₙₜᵣₐ represents the coordinates of the markers in the patient's intraoperative point cloud image, Tᵢₙᵢₜᵢₐᵢ represents the initial registration matrix for roughly aligning the three-dimensional model constructed based on preoperative images with the patient's point cloud,

$${T}_{Marker}^{Tra}$$

represents the marker transformation matrix, and is tracked in real time by the optical tracking system,

$${T}_{Marker}^{Tra}$$

represents the ultrasound calibration matrix, representing the relationship between the ultrasound device coordinate system and the tracking coordinate system, and Lₚᵣₑ represents the coordinates of the markers in the preoperative images.

In the present application, docking the three-dimensional model constructed based on preoperative images with the patient's point cloud can realize high-precision matching between preoperative images and intraoperative actual point clouds, thereby ensuring the accuracy of the ablation path, improving the controllability and safety of the treatment effect, and reducing intraoperative errors.

S803: On the basis of preliminary registration, an improved non-rigid ICP (Iterative Closest Point) algorithm is adopted to perform forward point matching by minimizing the Euclidean distance:

$$\vec{v} ( {v}_{i}^{{Q}^{′}} ) = arg {min}_{{v}_{j}^{P} ∈ P} {‖ {v}_{i}^{{Q}^{′}} - {v}_{j}^{P} ‖}_{2} , i = 1 , 2 , … , {N}_{Q} , j = 1 , 2 , … , {N}_{P} ;$$

Wherein, \vec{v} represents forward point matching,

$${v}_{i}^{{Q}^{′}}$$

represents the i-th data point in the deformed preoperative 3D model, argmin represents the variable value that minimizes the function value,

$${v}_{j}^{P}$$

represents the j-th data point in the patient's point cloud, P represents the patient's point cloud, ∥ ∥₂ represents the L2 norm calculation, N_{Q} represents the total number of data points in the 3D model constructed based on preoperative images, and N_{P} represents the total number of data points in the patient's point cloud.

It should be noted that by minimizing the Euclidean distance, each data point in the preoperative 3D model is matched with the closest point in the patient's point cloud, thereby ensuring the refinement of preliminary registration. This method can minimize errors caused by changes in the patient's posture or anatomical deformation, ensuring a high degree of consistency between the preoperative model and the intraoperative actual situation. Backward point matching is performed through global search:

$$\overleftarrow{v} ( {v}_{i}^{{Q}^{′}} ) = \{ {v}_{j}^{P} \vert \vec{v} ( {v}_{j}^{P} ) = {v}_{i}^{{Q}^{′}} \} , i = 1 , 2 , … , {N}_{Q} , j = 1 , 2 , … , {N}_{P} ;$$

Wherein, [symbol] represents backward point matching.

It should be noted that through global search, backward point matching can confirm whether each point in the patient's point cloud has been matched with a point in the preoperative 3D model, thereby further improving the matching accuracy. Backward matching ensures the correctness and consistency of registration, helps to further refine the matching results, and avoids incorrect matching.

In the present application, the combination of forward and backward point matching enables accurate alignment between preoperative images and the patient's intraoperative actual anatomy, improves registration accuracy, provides a reliable basis for the accurate planning of the ablation path, and ensures the safety and effectiveness of the treatment.

S804: Based on the forward point matching result and the backward point matching result, a registration cost function is set:

$$J = {\sum }_{{v}_{i}^{{Q}^{′}} ∈ {Q}^{′}} ( {ω}_{i} {‖ {v}_{i}^{{Q}^{′}} - {M}_{i} \vec{v} ( {v}_{i}^{{Q}^{′}} ) ‖}_{2} + {ω}_{j} {‖ {v}_{i}^{{Q}^{′}} - {M}_{j} \overleftarrow{v} ( {v}_{i}^{{Q}^{′}} ) ‖}_{2} ) + α {\sum }_{{v}_{t}^{P} , {v}_{j}^{P} ∈ P} {‖ {M}_{i} - {M}_{j} ‖}_{2}$$

Wherein, J represents the registration cost function, Q′ represents the data points in the deformed preoperative 3D model, ωᵢ represents the registration result parameter of the i-th data point in the deformed preoperative 3D model (ωᵢ is 1 if there is a corresponding data point for the i-th data point in the deformed preoperative 3D model, otherwise 0), Mᵢ represents the affine transformation matrix for transforming the i-th data point in the preoperative 3D model, ωⱼ represents the registration result parameter of the j-th data point in the patient's point cloud (ωj is 1 if there is a corresponding data point for the j-th data point in the patient's point cloud, otherwise 0), Mⱼ represents the affine transformation matrix for transforming the j-th data point in the patient's point cloud, and α represents the regularization coefficient.

In the present application, through the calculation of the registration cost function based on forward and backward point matching, the registration quality between the preoperative model and the patient's point cloud can be accurately evaluated. The registration cost function combines the Euclidean distance between matched points and the difference between transformation matrices to optimize the alignment accuracy of the model.

Furthermore, by introducing registration result parameters, the validity of matched points is flexibly considered, avoiding interference from mismatched points on the registration result. At the same time, the regularization term makes the registration process smoother, reduces unnecessary deformation, and ensures more accurate and stable registration results, thereby improving the accuracy of ablation path planning and navigation and providing reliable guidance for the operation.

S805: Performing iterative optimization with the goal of reducing the registration cost function to achieve accurate registration between the 3D model constructed based on preoperative images and the patient's point cloud.

S806: Mapping the optimal ablation path to the patient's point cloud.

S807: Performing ablation navigation according to the needle insertion position, angle, and depth determined after mapping. In the present application, after achieving accurate registration through iterative optimization, the optimal ablation path is mapped to the patient's point cloud to provide accurate guidance for ablation navigation. This can provide real-time and accurate path planning during the operation, reduce errors and complications, ensure that the ablation needle accurately reaches the target area, and thus improve the safety and effectiveness of the treatment.

The beneficial effects brought by the technical solutions provided by the embodiments of the present application include at least:

In the embodiments of the present application, hard constraint conditions, soft constraint conditions, the seagull optimization algorithm, and organ damage assessment are adopted, and the optimal ablation path is determined through multi-stage path optimization. It can handle cases involving multiple tumors, deeply located tumors, or tumors with complex locations, conduct a comprehensive risk assessment of the ablation path, and evaluate the potential damage of the path to surrounding tissues and organs. This ensures that the finally selected path not only has a low risk index but also avoids damage to normal organs to the greatest extent, guarantees the safety and effectiveness of the ablation process, and improves the accuracy of path selection and the efficiency of ablation navigation.

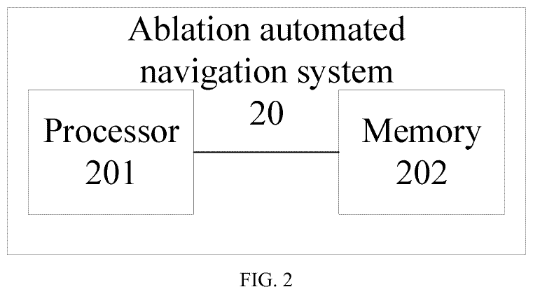

With reference to FIG. 2 in the specification, a schematic structural diagram of an ablation automated navigation system provided by the present application is shown. The present application also provides an ablation automated navigation system 20, applied to the above-mentioned ablation automated navigation method, including:

A processor 201;

A memory 202, where computer-readable instructions are stored on the memory 202. When the computer-readable instructions are executed by the processor 201, the ablation automated navigation method as described in the method embodiment is implemented.

The ablation automated navigation system 20 provided by the present application can execute the above-mentioned ablation automated navigation method and achieve the same or similar technical effects. To avoid repetition, details are not repeated herein. The beneficial effects brought by the technical solutions provided by the embodiments of the present application include at least:

In the embodiments of the present application, hard constraint conditions, soft constraint conditions, the seagull optimization algorithm, and organ damage assessment are adopted, and the optimal ablation path is determined through multi-stage path optimization. It can handle cases involving multiple tumors, deeply located tumors, or tumors with complex locations, conduct a comprehensive risk assessment of the ablation path, and evaluate the potential damage of the path to surrounding tissues and organs. This ensures that the finally selected path not only has a low risk index but also avoids damage to normal organs to the greatest extent, guarantees the safety and effectiveness of the ablation process, and improves the accuracy of path selection and the efficiency of ablation navigation.

It should be understood that the processor in the embodiments of the present application may be a Central Processing Unit (CPU), and the processor may also be other general-purpose processors, Digital Signal Processors (DSPs), Application-Specific Integrated Circuits (ASICs), Field-Programmable Gate Arrays (FPGAs) or other programmable logic devices, discrete gate or transistor logic devices, discrete hardware components, etc. A general-purpose processor may be a microprocessor, or the processor may also be any conventional processor, etc.

It should also be understood that the memory in the embodiments of the present application may be a volatile memory or a non-volatile memory, or may include both volatile and non-volatile memories. The non-volatile memory may be a Read-Only Memory (ROM), a Programmable Read-Only Memory (PROM), an Erasable Programmable Read-Only Memory (EPROM), an Electrically Erasable Programmable Read-Only Memory (EEPROM), or a flash memory. The volatile memory may be a Random Access Memory (RAM), which serves as an external high-speed cache. By way of example and not limitation, many forms of RAM are available, such as Static Random Access Memory (SRAM), Dynamic Random Access Memory (DRAM), Synchronous Dynamic Random Access Memory (SDRAM), Double Data Rate Synchronous Dynamic Random Access Memory (DDR SDRAM), Enhanced Synchronous Dynamic Random Access Memory (ESDRAM), Synchronous Link Dynamic Random Access Memory (SLDRAM), and Direct Rambus Random Access Memory (DR RAM).

The above-mentioned embodiments may be implemented in whole or in part by software, hardware (such as circuits), firmware, or any other combination. When implemented using software, the above-mentioned embodiments may be implemented in whole or in part in the form of a computer program product. The computer program product includes one or more computer instructions or computer programs. When the computer instructions or computer programs are loaded or executed on a computer, the processes or functions according to the embodiments of the present application are generated in whole or in part. The computer may be a general-purpose computer, a dedicated computer, a computer network, or other programmable devices. The computer instructions may be stored in a computer-readable storage medium or transmitted from one computer-readable storage medium to another computer-readable storage medium. For example, the computer instructions may be transmitted from a website, computer, server, or data center to another website, computer, server, or data center via a wired (such as infrared, radio, microwave, etc.) method. The computer-readable storage medium may be any available medium that can be accessed by a computer or a data storage device such as a server or a data center that includes one or more available medium sets. The available medium may be a magnetic medium (such as a floppy disk, a hard disk, a magnetic tape), an optical medium (such as a DVD), or a semiconductor medium (such as a solid-state drive).

It should be understood that the term “and/or” in this document is only a kind of association relationship describing associated objects, indicating that there may be three kinds of relationships. For example, A and/or B may mean: A exists alone, A and B exist simultaneously, or B exists alone, where A and B may be singular or plural. In addition, the character “/” in this document generally indicates that the associated objects before and after are in an “or” relationship, but it may also indicate an “and/or” relationship, which can be understood with reference to the specific context.

In the present application, “at least one” means one or more, and “a plurality” means two or more. The expression “at least one of the following” or similar expressions refers to any combination of these items, including a single item (individual) or a plurality of items (plurality). For example, at least one of a, b, or c may represent: a, b, c, a-b, a-c, b-c, or a-b-c, where a, b, c may be single or plural.

It should be understood that in various embodiments of the present application, the sequence numbers of the above processes do not mean the order of execution. The order of execution of each process should be determined by its function and internal logic, and should not constitute any limitation on the implementation process of the embodiments of the present application.

Those skilled in the art may realize that the units and algorithm steps of the examples described in conjunction with the embodiments disclosed herein can be implemented by electronic hardware or a combination of computer software and electronic hardware. Whether these functions are implemented in hardware or software depends on the specific application and design constraints of the technical solution. A professional technician may use different methods to implement the described functions for each specific application, but such implementation should not be considered to be beyond the scope of the present application.

Those skilled in the art can clearly understand that for the convenience and conciseness of description, the specific working processes of the above-described devices, apparatuses, and units may refer to the corresponding processes in the foregoing method embodiments, and details are not repeated herein.

In several embodiments provided in the present application, it should be understood that the disclosed devices, apparatuses, and methods may be implemented in other ways. For example, the above-described apparatus embodiments are only illustrative. For example, the division of the units is only a logical function division. In actual implementation, there may be other division methods. For example, multiple units or components may be combined or integrated into another device, or some features may be omitted or not executed. In addition, the displayed or discussed mutual coupling or direct coupling or communication connection may be implemented through some interfaces. The indirect coupling or communication connection between the apparatuses or units may be implemented in electrical, mechanical, or other forms.

The units described as separate components may or may not be physically separate, and the components displayed as units may or may not be physical units, that is, they may be located in one place, or may be distributed on multiple network units. Some or all of the units may be selected according to actual needs to achieve the objectives of the solutions of the embodiments.

In addition, each functional unit in each embodiment of the present application may be integrated into one processing unit, or each unit may exist alone physically, or two or more units may be integrated into one unit.

If the function is implemented in the form of a software functional unit and sold or used as an independent product, it may be stored in a computer-readable storage medium. Based on this understanding, the essence of the technical solution of the present application, or the part that contributes to the prior art, or part of the technical solution, may be embodied in the form of a software product. The computer software product is stored in a storage medium and includes several instructions for enabling a computer device (which may be a personal computer, a server, or a network device, etc.) to execute all or part of the steps of the method described in each embodiment of the present application. The aforementioned storage medium includes: a USB flash drive, a mobile hard disk, a Read-Only Memory (ROM), a Random Access Memory (RAM), a magnetic disk, an optical disc, and other media that can store program codes.

An embodiment of the present application provides a computer-readable storage medium, on which a computer program is stored. When the program is executed by a processor, the ablation automated navigation method as described in the method embodiment is implemented. The computer-readable storage medium provided by the present application can implement the steps and effects of the ablation automated navigation method in the above method embodiment. To avoid repetition, details are not repeated herein.

The beneficial effects brought by the technical solutions provided by the embodiments of the present application include at least:

In the embodiments of the present application, hard constraint conditions, soft constraint conditions, the seagull optimization algorithm, and organ damage assessment are adopted, and the optimal ablation path is determined through multi-stage path optimization. It can handle cases involving multiple tumors, deeply located tumors, or tumors with complex locations, conduct a comprehensive risk assessment of the ablation path, and evaluate the potential damage of the path to surrounding tissues and organs. This ensures that the finally selected path not only has a low risk index but also avoids damage to normal organs to the greatest extent, guarantees the safety and effectiveness of the ablation process, and improves the accuracy of path selection and the efficiency of ablation navigation.

The above descriptions are only specific implementations of the present application, but the protection scope of the present application is not limited thereto. Any person skilled in the art can easily think of changes or substitutions within the technical scope disclosed by the present application, which should be covered within the protection scope of the present application. Therefore, the protection scope of the present application should be subject to the protection scope of the claims. The following points need to be noted:
(1) The drawings of the embodiments of the present application only involve the structures related to the embodiments of the present application, and other structures can refer to the general design.
(2) For clarity, in the drawings used to describe the embodiments of the present application, the thickness of layers or regions is enlarged or reduced, that is, these drawings are not drawn according to actual scales. It can be understood that when an element such as a layer, film, region, or substrate is referred to as being “on” or “under” another element, the element can be “directly” on or “under” the other element, or an intermediate element may exist.
(3) Without conflict, the embodiments of the present application and the features in the embodiments can be combined with each other to form new embodiments.

The above descriptions are only specific implementations of the present application, but the protection scope of the present application is not limited thereto. The protection scope of the present application should be subject to the protection scope of the claims.

What is claimed is:

1. An ablation automated navigation system, comprising:
a processor;
a memory;
where computer-readable instructions are stored on the memory, and when the computer-readable instructions are executed by the processor to achieve a following ablation automated navigation method:
S1: setting hard constraint conditions and soft constraint conditions for an ablation path;
S2: based on the hard constraint conditions, initializing multiple preliminary ablation paths that meet the hard constraint conditions;
S3: based on the soft constraint conditions, calculating a risk index of each of the preliminary ablation paths;
S4: sorting each of the preliminary ablation paths in ascending order of the risk index, and selecting a first preset number of preliminary ablation paths with top rankings as initial solutions of a seagull optimization algorithm;
S5: optimizing the ablation path through the seagull optimization algorithm to determine multiple candidate ablation paths;
S6: sorting each of the candidate ablation paths in ascending order of fitness, and selecting a second preset number of candidate ablation paths with top rankings for organ damage assessment;
S7: determining an optimal ablation path from the candidate ablation paths according to an organ damage assessment result;
S8: performing ablation navigation according to a needle insertion position, angle and depth determined by the optimal ablation path;
a fitness function of the seagull optimization algorithm is specifically:
$$F ( l ) = {λ}_{d} {d}_{l} + {λ}_{a} {a}_{l} - {λ}_{h} {h}_{l} - {λ}_{h} {θ}_{l} ;$$
wherein, F( ) represents the fitness function, l represents the ablation path, dₗ represents a distance between the ablation path l and a risk structure, λ_{d} represents a weight coefficient of a risk structure term, al represents a puncture angle of the ablation path l, λₐ represents a weight coefficient of a puncture angle term, hₗ represents a puncture depth of the ablation path l, λₕ represents a weight coefficient of a puncture depth term, θₗ represents a number of CT layers through which the ablation path l passes, and λ_{θ} represents a weight coefficient of a term of the number of CT layers passed through;
wherein, S5 specifically comprises:
S501: sorting each preliminary ablation path in ascending order of the risk index, selecting the first preset number of preliminary ablation paths with top rankings as initial solutions of the seagull optimization algorithm, initializing seagull individuals, wherein each seagull individual represents a set of feasible model parameters, each seagull individual is composed of multiple dimensional components, and each component represents a model parameter;
S502: in a global search phase, avoiding collision and move towards the optimal individual:
$${D}_{i}^{t} = \vert {C}_{i}^{t} + {M}_{i}^{t} \vert$$
$${C}_{i}^{t} = A \times {P}_{i}^{t}$$
$${M}_{i}^{t} = B \times ( {P}_{b e s t}^{t} - {P}_{i}^{t} ) ;$$
wherein
$${D}_{i}^{t}$$
represents a position of an i-th seagull individual after the global search phase in a t-th iteration,
$${C}_{i}^{t}$$
represents a position of the i-th seagull individual after anti-collision processing in the t-th iteration, A represents the control factor,
$${P}_{i}^{t}$$
represents a position of the i-th seagull individual in the t-th iteration,
$${M}_{i}^{t}$$
represents a displacement of the i-th seagull individual moving towards an optimal individual in the t-th iteration, B represents a search balance factor, and
$${P}_{b e s t}^{t}$$
represents a position of an optimal individual in the t-th iteration;
$$A = [ 1 - lg ( \frac{9 t}{T} ) ] \times {f}_{c} ;$$
wherein t represents a current number of iterations, T represents a maximum number of iterations, and f_{c} represents a linear descent frequency;
$$B = 2 {A}^{2} {r}_{1} ;$$
wherein r1 represents a random number between 0 and 1;
S503: in a local search phase, generating a random number r2, and according to a random number r2, selecting in parallel between a spiral search strategy and an encirclement strategy to perform displacement in a spiral motion manner:
$$\{ \begin{matrix}{E}_{i}^{t} = {D}_{i}^{t} x y z + {P}_{b e s t}^{t} & {r}_{2} \leq 0.5 \\ {E}_{i}^{t} = ( {D}_{i}^{t} + {P}_{b e s t}^{t} ) x y z & {r}_{2} > 0.5\end{matrix}$$
$$x = r sin η$$
$$x = r cos η$$
$$z = r η$$
$$r = u {e}^{η v} ;$$
wherein
$${E}_{i}^{t}$$
represents a position of the i-th seagull individual after spiral motion in the t-th iteration, x represents a spiral flight coefficient in a x direction, y represents a spiral flight coefficient in a y direction, z represents a spiral flight coefficient in a z direction, r represents a radius of a spiral flight trajectory, η represents a random number between 0 and 2π, u and v represent spiral constants, and e represents a natural constant;
S504: performing mutation operation on each seagull individual:
$${V}_{i}^{t} = {E}_{i}^{t} + γ ( {P}_{b e s t}^{t} - {P}_{r} ) ;$$
wherein
$${V}_{i}^{t}$$
represents a position of the i-th seagull individual after mutation in the t-th iteration, Pr represents a random individual, and γ represents a adaptive scale factor;
$$γ = {γ}_{max} - ( {γ}_{max} - {γ}_{min} ) sin [ \frac{π}{2} {( \frac{t}{T} )}^{2} ] ;$$
wherein γmax represents a maximum scale factor, γmin represents a minimum scale factor, and sin represents a sine function;
S505: judging whether a fitness value of a mutated position is greater than that of a position before mutation; if yes, replacing the position before mutation with the mutated position; otherwise, keeping the position before mutation unchanged;
S506: updating the fitness value of each seagull individual and the global optimal individual;
S507: judging whether a current number of iterations reaches a maximum number of iterations; if yes, outputting a model parameter set represented by the seagull individual with a highest current fitness; otherwise, returning to continue iteration;
wherein S8 specifically comprises:
S801: pasting multiple markers on a surface of a patient's abdominal skin;
S802: based on multiple markers, performing preliminary registration between a three-dimensional model constructed based on preoperative images and the patient's point cloud;
S803: on a basis of preliminary registration, adopting an improved non-rigid ICP algorithm to perform forward point matching by minimizing an Euclidean distance;
performing backward point matching through global search;
S804: setting a registration cost function based on a forward point matching result and a backward point matching result:
$$J = {\sum }_{{v}_{i}^{{Q}^{′}} ∈ {Q}^{′}} ( {ω}_{i} {‖ {v}_{i}^{{Q}^{′}} - {M}_{i} \vec{v} ( {v}_{i}^{{Q}^{′}} ) ‖}_{2} + {ω}_{j} {‖ {v}_{i}^{{Q}^{′}} - {M}_{j} \overleftarrow{v} ( {v}_{i}^{{Q}^{′}} ) ‖}_{2} ) + α {\sum }_{{v}_{i}^{P} , {v}_{j}^{P} ∈ P} {‖ {M}_{i} - {M}_{j} ‖}_{2} ;$$
wherein J represents a registration cost function, Q′ represents data points in a deformed preoperative three-dimensional model, ωᵢ represents a registration result parameter of the i-th data point in a deformed preoperative three-dimensional model, ωᵢ is 1 when there is a corresponding data point for the i-th data point in the deformed preoperative three-dimensional model, otherwise 0, Mᵢ represents a affine transformation matrix for transforming the i-th data point in the deformed preoperative three-dimensional model, ω₁ represents a registration result parameter of a j-th data point in the patient's point cloud, ωⱼ is 1 when there is a corresponding data point for the j-th data point in the patient's point cloud, otherwise 0, Mⱼ represents an affine transformation matrix for transforming the j-th data point in the patient's point cloud, and a represents a regularization coefficient;
S805: performing iterative optimization with a goal of reducing a registration cost function to accurately register a three-dimensional model constructed based on preoperative images with the patient's point cloud;
S806: mapping the optimal ablation path to the patient's point cloud;
S807: performing ablation navigation according to a needle insertion position, angle and depth determined after mapping.
